(19) United States
(12) Patent Application Publication
(10) Pub. No.: US 2007/0282217 A1
(43) Pub. Date: Dec. 6, 2007

(54) Methods & systems for intraoperatively monitoring nerve & muscle frequency latency and amplitude

(76) Inventors: William J. McGinnis, Cincinnati, OH (US); Scott A. Metrick, Glenview, IL (US)

Correspondence Address: LADAS & PARRY LLP, 224 SOUTH MICHIGAN AVENUE, SUITE 1600, CHICAGO, IL, 60604, US

(21) Appl. No.: 11/445,122

(22) Filed: Jun. 1, 2006

(51) Int. Cl. A61B 5/04

(52) U.S. Cl. 600/546; 600/554

(57) ABSTRACT

Methods and systems are provided for neurophysiological assessment, specifically nerve and nerve root conduction frequencies, latencies and amplitudes, with respect to surgical intervention and insult. Real-time trends in waveforms are captured, and warnings of pathological changes reported, displayed and audibilized.

### FIELD OF THE INVENTION

This invention relates to the field of neurophysiology, specifically electrophysiological evaluation of evoked nerve conduction latencies and amplitudes, electromyograph activity and compound muscle action potentials, as well as spontaneous electromyography and nerve action potentials in a subject.

### BACKGROUND OF THE INVENTION

Mixed nerve somatosensory evoked potentials (SSEP) are assessed neurophysiologically for latency and amplitude measurements that reflect mixed nerve (both sensory and motor fiber) function, are well documented in the medical literature as neurophysiologic peripheral representations of spinal cord function, are rather robust in nature and easily obtained from peripheral stimulation sites. SSEP responses are averaged and a mean mathematical representation is presented as an “evoked response” or “evoked potential.” Generally, mixed nerve SSEP are robust and easily obtained from peripheral stimulation sites, and their use is well established clinically for evaluating the electrophysiological presentation in subjects with neurological symptoms. Anatomically innervated by multiple overlapping nerve roots, SSEP assess mixed nerve function and cannot be used specifically to identify problems found with individual nerve roots. SSEP may be normal in subjects having significant pathology. By contrast, dermatomal evoked potentials (DSSEP), the physiological representation of specific nerve root function are technically demanding and difficult to obtain.

Although obtaining DSSEPs is non-invasive, and relatively inexpensive, the technique is technically demanding, and reproducible results are difficult to obtain. The literature identifies the primary recording site for a dermatomal response as being over the somatosensory cortex. However, signals from the cortex are known to be ambiguous at best in both awake and in anaesthetized subjects.

Furthermore, the incidence of occurrence of post-operative sciatic nerve palsy following revision total hip arthroplasty (RTHA) surgery is reported to be 1-15%. The preponderance of electrophysiological intraoperative data assessing sciatic nerve function during these procedures address assessment of cortically elicited somatosensory evoked potentials latency with posterior tibal nerve stimulation. However, besides being anesthesia dependant these potentials may lack sensitivity and specificity with regard to neurophysiological insult to contributing innervation, peroneal and posterior tibial.

Owen et al, (Spine vol. 18, No. 6, pgs 748-754 (1993)) in studying the differences in the levels of the DSSEP and nerve root involvement, report variable results in the peripheral innervations patterns of the dorsal nerve roots in the cervical and lumbar spine. U.S. Pat. No. 5,338,587 addressed the lack of reproducibility of responses detected at the cerebral cortex through static comparisons of transport times (latency) of signals from different stimulating electrodes.

It has been surprisingly found that superior and robust DSSEP waveforms may be recorded at a subcortical recording site. Reproducible high-confidence DSSEP data would be a considerable advance in the field.

It would also be highly advantageous to clinicians and surgeons alike to be able to compare evoked potentials in real-time and perform real-time comparisons between waveforms while they are being recorded during neurophysiological assessment, particularly intraoperatively. There also remains a need for an intraoperative monitoring technique for reducing postoperative nerve palsy or damage associated with surgeries involving at-risk nerves. A way of segmentally monitoring an at-risk nerve function would be of importance in the field.

### SUMMARY OF THE INVENTION

In accordance with the present invention, these and other problems are solved by the methods and systems described herein for monitoring and comparing one or more neurophysiological responses in a mammalian subject, and more specifically for (a) obtaining, amplifying and storing in a buffer time-locked frequency, a latency and an amplitude waveform signal; (b) automatically digitally converting the waveform signal and assigning a set of numeric values for the waveform frequency, waveform absolute amplitude and waveform absolute latency; (c) replicating (a) and (b) to obtain a series of replicated digitally assigned waveform data for the given monitoring site; and (d) mathematically conditioning the series of replicated digitally assigned waveform data to obtain a validated mean value for the series of replicated digitally assigned waveform data. In preferred embodiment, the invention provides for performing a series of further trials in the above-described manner, and for serially comparing and evaluating in real-time the changes in the serially obtained waveform data, and for saving and reporting the comparisons and changes as a function of time.

In the instant approach, it should be understood that the monitored neurophysiological response may comprise spontaneously occurring activity, or mechanically elicited responses or electrically elicited responses, and for example, may be selected from the group consisting of SSEP, DSSEP, spontaneously occurring electromyography, sEMG, mechanically elicited electromyography, mEMG, spontaneously occurring nerve action potentials, sNAP, mechanically elicited nerve action potentials, mNAP, and electrically elicited nerve action potentials, eNAP, and electrically elicited compound muscle action potentials, eCMAP.

It should be also understood that the approach outlined in (a) to (d) above is intended to apply to not just one monitoring site in a subject but to a second or to a plurality of different stimulation sites.

In a preferred embodiment, the waveform data is compared, throughout steps (a) to (d) above, in real-time as a function of time, comprising performing a series of recording trials in the above-described manner and serially obtaining, comparing and evaluating in real-time the changes in the waveform data; and saving same as a function of time.

In another aspect of the invention, a system is provided for comparing and assessing neurophysiological activity at one or more recording sites on a mammalian subject during a procedure, the system comprising: computer-readable media and data storage means having encoded instructions for executing: receiving and recording a time-locked waveform signal at a recording site on the subject, amplifying the recorded waveform signal, recording and saving a frequency value, a latency value, and an amplitude value for the amplified waveform signal, automatically digitally converting the frequency, latency and amplitude of the waveform signal; assigning numeric values for the frequency, for the absolute amplitude and the absolute latency of the waveform, replicating these steps to obtain a series of replicated digitally assigned waveform data for the given recording site, and mathematically conditioning the replicated digitally assigned waveform data to obtain a set of validated mean values for the waveform data. In one preferred embodiment, hardware and software means are provided for comparing of the validated mean values with protocol-specific and/or subject-specific normal data, wherein the comparison is assessed and the deviations of the waveform data from normal noted. In another preferred embodiment, hardware and software means are provided for performing a series of further trials in the manner of the above described and serially comparing and evaluating in real-time the changes in the waveform data, and for saving the comparisons and changes as a function of time.

In yet a further aspect, the invention comprises a software-driven data storage medium readable by a computing system and encoding a computer program for executing a computer process for buffering and comparing neurophysiological spontaneous and elicited responses in a data storage system, the computer program comprising instructions for carrying out the inventive approach as described herein. In yet another aspect, the invention comprises a computer data signal embodied in a carrier wave by a computing system and encoding a computer program for executing a computer process for buffering and comparing evoked potential responses in a data storage system including a processor, memory, and storage devices, the computer program comprising instructions for carrying out the inventive approach as described herein.

In a preferred embodiment, evoked DSSEPs are elicited from a stimulating module (which can be an electrode, a pin, a tab, a probe, or other suitable means for providing a stimulus) at a dermatomal site on the subject. In another preferred embodiment, neurological responses are obtained from a subject and are recorded over the posterior cervical spine of the subject. In yet another aspect of the invention, recorded evoked potentials are correlated with recorded electromyography obtained from the subject.

In a further embodiment, the invention is directed to monitoring an at-risk nerve during a surgical procedure, comprising: (a) recording a baseline spontaneously occurring electromyography activity (sEMG); then serially, throughout the procedure, recording a plurality of mechanically elicited electromyography activity (mEMG); and therefrom obtaining a plurality of real-time mEMG/sEMG comparisons between the serially obtained mEMG recordings and the sEMG recording; (b) recording a baseline spontaneously occurring nerve action potential (sNAP); then serially, throughout the procedure, recording a plurality of mechanically elicited nerve action potentials (mNAP); and therefrom obtaining a plurality of real-time mNAP/sNAP comparisons between the serially obtained mNAP recordings and the sNAP recording; (c) serially, throughout the procedure, electrically stimulating a (sciatic) nerve portion and following each act of stimulating, obtaining a plurality of electrically elicited compound muscle action potential (eCMAP) recordings, and a plurality of electrically elicited compound nerve action potential (eNAP) recordings; and therefrom obtaining a plurality of real-time eCMAP/eCMAP comparisons between the serially obtained eCMAP recordings and a plurality of real-time eNAP/eNAP comparisons between the serially obtained eNAP recordings, wherein the obtained mEMG, sEMG, mNAP, sNAP, eCMAP, eNAP recordings, and the mEMG/sEMG, mNAP/sNAP, eCMAP/eCMAP and eNAP/eNAP comparisons are displayed by means of one or more of a plurality of real-time trend displays, and a processor adapted and configured to receive and store in a plurality of real-time data acquisition buffers the obtained mEMG, sEMG, mNAP, sNAP, eCMAP, eNAP recordings, and receive and store in a plurality of real-time comparison buffers the mEMG/sEMG, mNAP/sNAP, eCMAP/eCMAP and eNAP/eNAP comparisons, said processor being in communication with said real-time trend displays, and wherein said real-time trend displays comprise: one or a plurality of baseline data windows wherein one or more baseline data are displayed; one or more amplitude waveform windows wherein one or more amplitude waveforms are displayed with respect to time; one or a plurality of latency waveform windows wherein one or more latency waveforms are displayed with respect to time; one or a plurality of frequency waveform windows wherein one or more frequency waveforms are displayed with respect to time; and one or a plurality of real-time digital value analysis windows, wherein recorded analog data converted to digital values are displayed with respect to time, said digital value analysis windows providing a plurality of evaluations in real-time.

### BRIEF DESCRIPTION OF THE DRAWINGS

FIG. 1 shows the upper extremities stimulation sites.

FIG. 2 shows the left hand stimulation sites.

FIG. 3A shows the lower extremities stimulation sites.

FIG. 3B shows the foot stimulation sites.

FIG. 4 shows the ulnar nerve stimulation site.

FIG. 5 shows the posterior cervical recording site.

FIG. 6 shows the peroneal and posterior tibial stimulation sites.

FIG. 7 shows the lumbar potential recording site.

FIGS. 8A-D show sample waveforms for the C5, C6, C7 and C8 dermatomes, respectively.

FIG. 9 shows a sample waveform for a mixed median response.

FIG. 10 illustrates schematically the methods and systems of the present invention.

FIG. 11 is diagram of the connection box.

FIG. 12 is a diagram of the stimulus site selector switchbox.

FIGS. 13A-C illustrate electromyograph recording sites for intraoperative verification of root nerves, respectively, 13A is an anterior view, 13B is an upper posterior view, 13C is a lower posterior view.

FIG. 14 depicts a flowchart of operations for obtaining real-time validated mean values for digitally assigned waveform data, and obtaining comparisons in real-time.

FIG. 15 depicts a flowchart of operations for obtaining, reporting and comparing in real-time, EMG, NAP and CMAP recordings for monitoring at-risk nerve function.

### DETAILED DESCRIPTION OF THE PREFERRED EMBODIMENTS

### Definitions

Somatosensory Evoked Potentials (SSEP)

Somatosensory evoked potentials (SSEP) mixed nerve responses are typically elicited by stimulation of mixed nerves at various anatomical locations, such as wrist (median), elbow (ulnar), knee (peroneal) and ankle (posterior tibial). The evoked signals are electrical impulses that are recorded from electrodes placed over the crown of the subject's head at the cerebral cortex.

Dermatomal Somatosensory Evoked Potentials (DSSEP)

Dermatomal somatosensory evoked potentials (DSSEP) are the physiologic representation of specific nerve root function, used to evaluate sensory input from individual nerve roots. Conventional practice utilizes recording electrodes over the somatosensory cortex on the head, with a subcortical potential recorded over the posterior cervical spine only as an adjunct site. In dermatomal somatosensory evoked potentials, specific skin sites are stimulated by a mild electrical stimulus which causes an evoked response that travels through the nerve, nerve root and spinal cord to the brain. The time (latency) taken for the evoked response to travel through the nervous system can be measured and compared to a control. If the evoked response travel time is slowed, then nerve root pathology is likely. A single dermatosensory evoked potential test procedure takes less than one minute and is repeated to assess multiple nerve roots appropriate to the particular situation.

It has been found by the inventors that DSSEPs are particularly helpful in evaluating individuals with spine and limb symptoms (i.e. pain, numbness and/or weakness). A common example is in “sciatica” of a “pinched nerve” in which a spine pain may radiate into a limb. An MRI may reveal multilevel changes and a diagnostician is uncertain which level is relevant to the subject's symptoms. DSSEPs can help make this distinction.

DSSEPs have also been found by the inventors to be helpful during surgical spinal decompression procedures. DSSEPs are performed continuously throughout the procedure and are compared in real-time to the subject's preoperative DSSEP. If an adequate decompression is accomplished by the surgeon, the previously delayed DSSEP may be seen to revert to normal or speed up which provides immediate reassurance to the surgeon. Alternatively, if the surgeon is operating near a nerve root and the DSSEP becomes delayed, then the surgeon may be alerted to the potential for injury to the nerve root by the surgery.

Electromyogram (EMG)

Measurement of an electromyogram (EMG) provides an additional neurophysiologic parameter to assess neurophysiologic function in both the clinical and intraoperative setting, founded in the fact that nerve roots distribute to both dermatomes and to muscles or myotomes. While DSSEP and the SSEP provide information about the transmission of an electrical signal from the peripheral nervous system to the central nervous system, recording and evaluating EMG provides information about the innervation of a particular muscle (efferent) from the central nervous system to the muscle, as well as providing information about the irritability and conductivity of the muscle itself. The electromyography referred to herein may be spontaneous(free-running) electromyography (sEMG) or mechanically elicited electromyography (mEMG) resulting from mechanical interference (surgical intervention or insult

Compound Muscle Action Potential (CMAP)

CMAP activity is the summation of nearly synchronous muscle fiber action potentials recorded from a muscle, commonly produced by stimulation of the nerve supplying the muscle either directly or indirectly. The CMAP activity referred to herein may for example be electrically elicited from the specific muscle innervated by the nerve, such as for example, the quadriceps, tibialis anterior, gastrocnemius and extensor hallucis longus (EHL) musculature. Baseline-to-peak amplitude, duration and latency of the negative phase is recorded.

Nerve Action Potential (NAP)

NAP activity is obtained from needle electrodes placed near the medial and lateral femoral cutaneous, peroneal and posterior tibial nerves. NAP activity is the summation of nearly synchronous nerve fiber action potentials recorded from a nerve trunk, commonly produced by stimulation of the nerve directly or indirectly. The nerve fiber action can be sensory, motor or mixed nerve. The nerve action potential referred to herein may be spontaneous(free-running) nerve action potential (sNAP) or mechanically elicited nerve action potential (mNAP) resulting from mechanical interference (surgical intervention or insult).

The inventive methods and systems are based upon the recording the transit time and amplitude of the charge through the body which is represented by waveforms. When a site is stimulated with an electrical stimulus, the time taken in milliseconds (ms) for the stimulus to travel to the recording electrode is recorded, multiple stimuli from the same stimulation locus are averaged, and comparisons made between validated numeric representations of the waveform. The latency of the waveforms is specifically considered using signal enhancement of distributed waveforms. For real-time evaluation, recording is performed repeatedly upon a subject to elicit serial evoked responses from multiple stimulation sites.

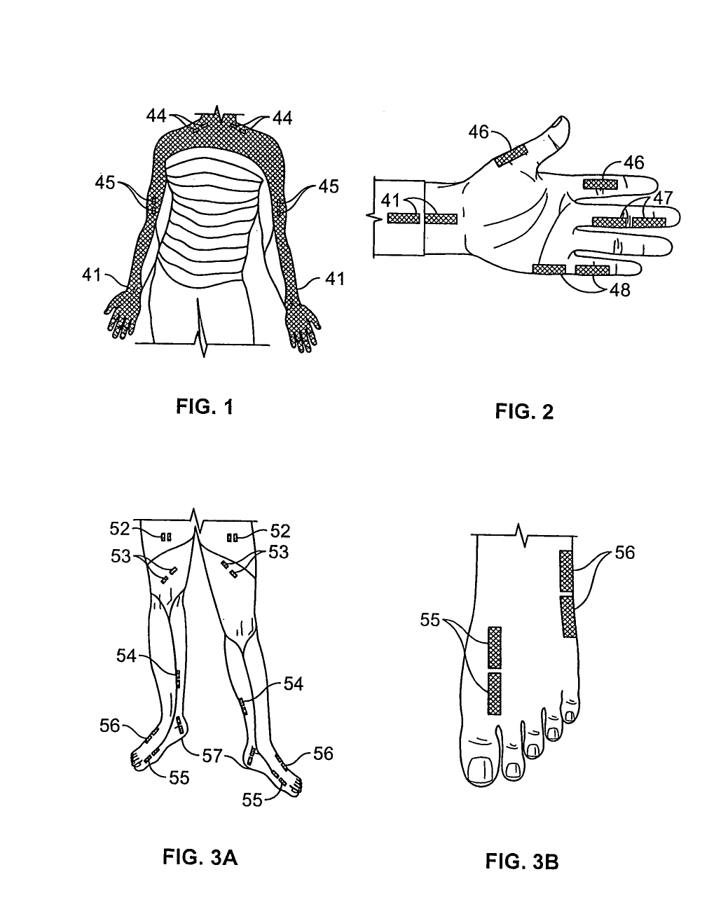

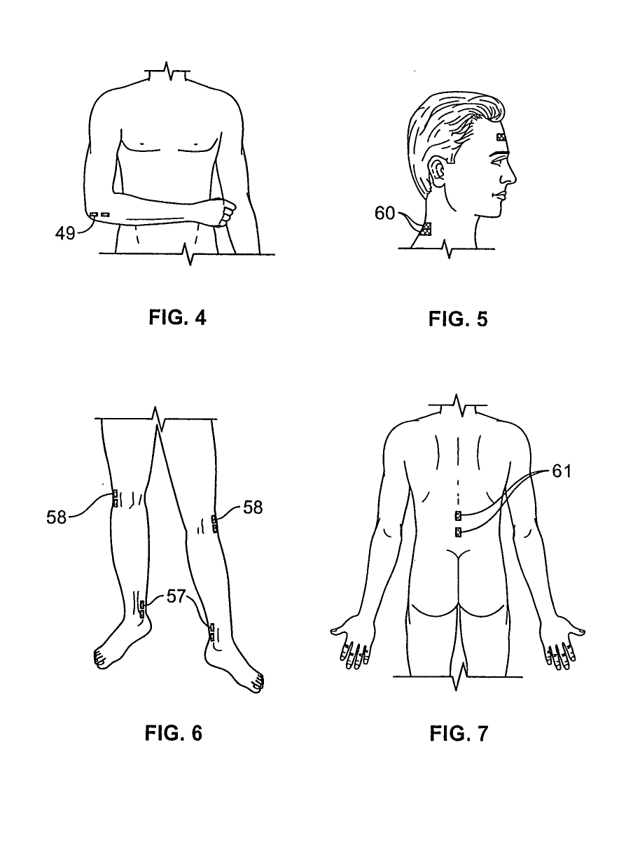

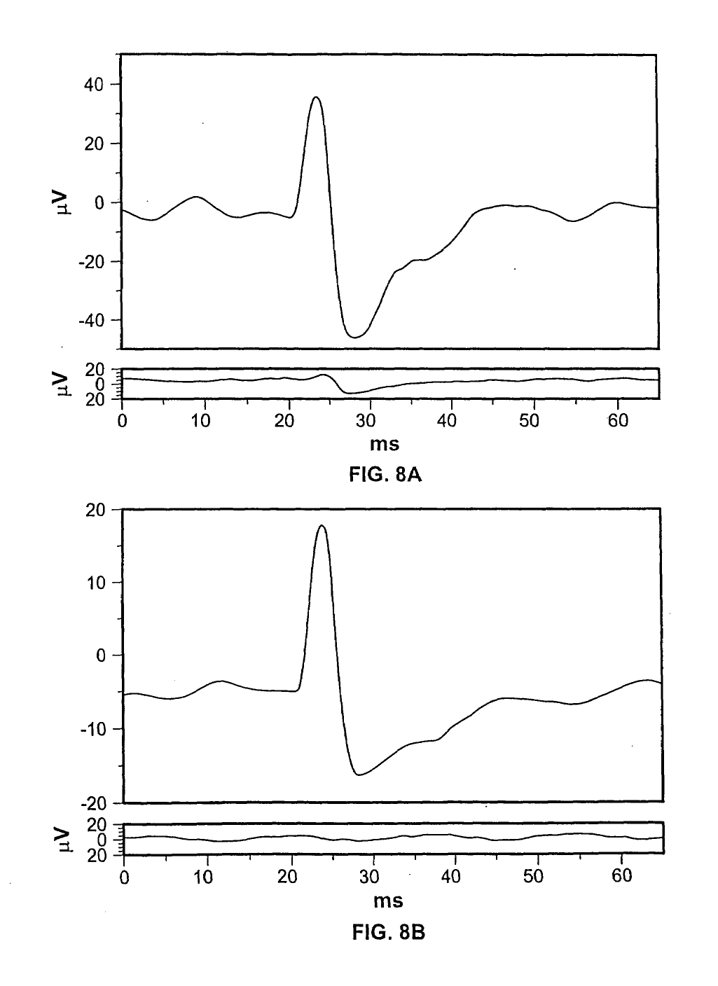

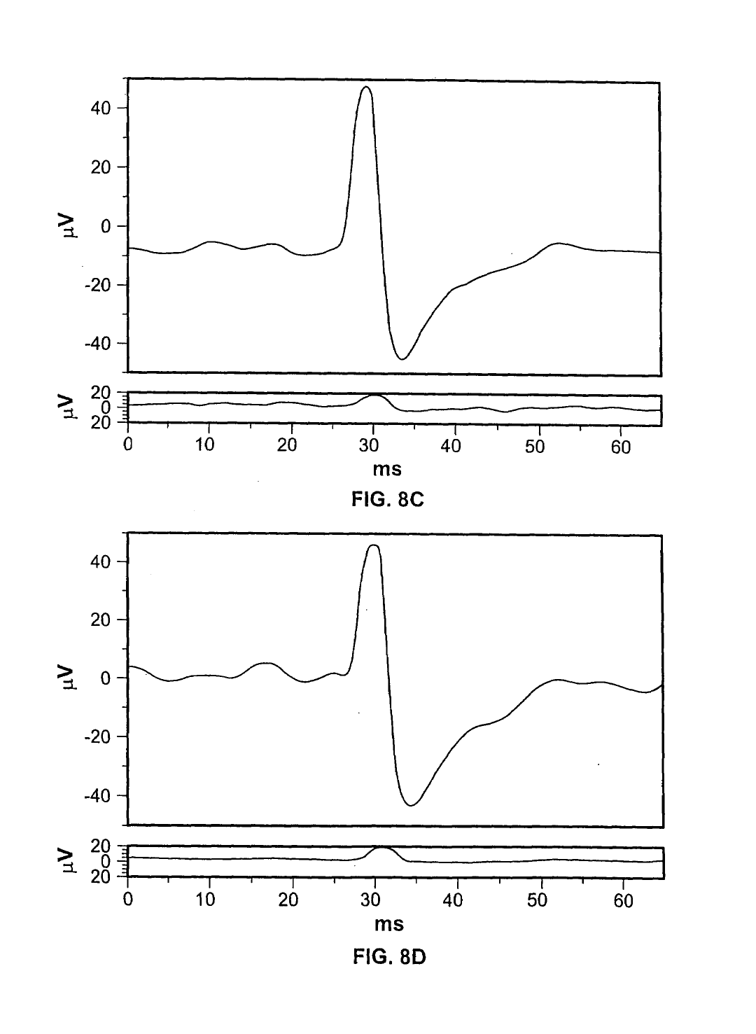

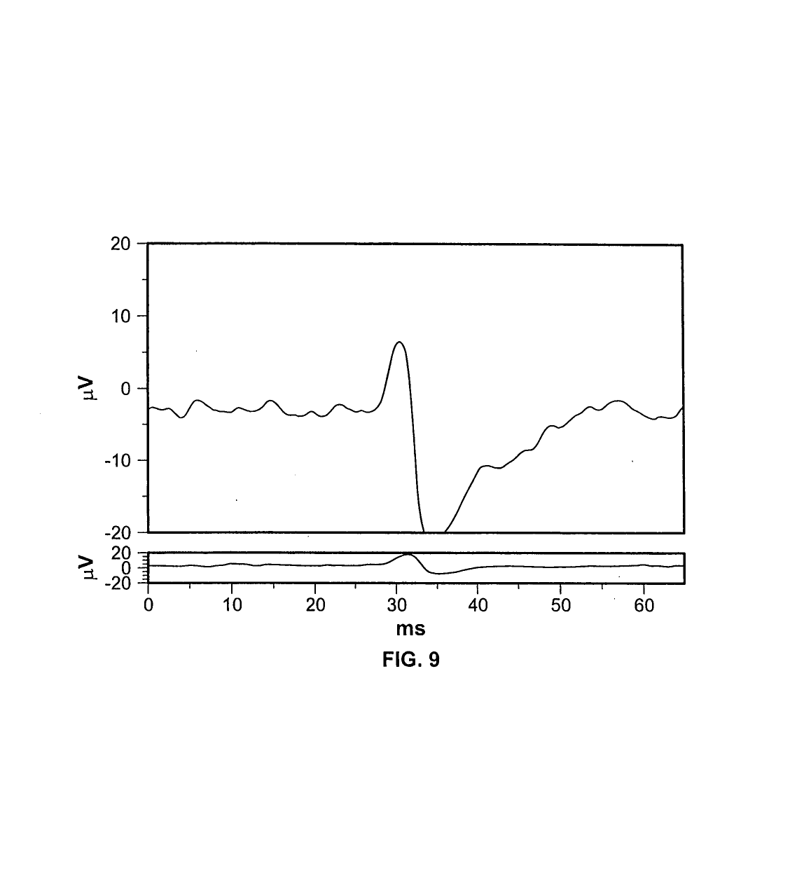

Typical stimulation sites used in the present invention are shown in FIGS. 1 and 2 (upper extremities), FIGS. 3A and 3B (lower extremities), FIG. 4 (ulnar stimulation site, and FIG. 6 (posterior tibial stimulation site). FIGS. 1 and 2 illustrate the bilateral stimulation sites in the upper extremities at C4 (44), C5 (45), C6 (46), C7 (47) and C8 (48), and via the mixed median (41) (referring to reference numbers on the drawings). FIGS. 3A and 3B similarly show the lower extremities stimulation sites, L2 (52), L3 (53), L4 (54), SI (56), L5 (55) and the posterior tibial stimulation sites (57). FIG. 4 shows the ulnar nerve stimulation site (49). The position of the posterior cervical recording site is shown in FIG. 5 by reference number 60. FIG. 6 shows the positions of the peroneal stimulation sites (58) with the posterial tibial stimulation sites (57). The position of the lumbar potential recording sites is shown in FIG. 7 (61). FIGS. 8A-D and 9 show sample waveforms for C5, C6, C7, C8, and for mixed median response, respectively.

In trying to optimize the technical recording of such responses, it was discovered that significant improvement in the quality and replication of SSEP and DSSEP are achieved by the use of low stimulus intensity, greater stimulus duration, larger surface area contacts and a decreased improved amplifier signal-to-noise ratio. Stimulus artifact is reduced by employing longer stimulus durations and using thresholds well below motor response to reduce antidromic propagation.

To elicit a dermatomal response, an electrical current is applied to the skin which produces an electrical depolarization in small nerve fibers at a specific dermatomal site. Thereby, an afferent volley of depolarization passes orthodromicly through the nerve, nerve root entry and spinal cord to the somatosensory cortex. Given the small nature of the end fibers, the more fibers that can be recruited, the greater the amplitude of the mathematical representation of the individual roots innervation. It is important therefore to recruit a large number of sensory end fibers without exceeding threshold to elicit motor involvement. A robust evoked potential is achieved therefore when a greater contact area for the stimulating modules is used. In the invention, it should be born in mind that a stimulus may be provided by any suitable means for providing a stimulus, such as for example an electrode, a pin, a tab, a probe. The greater the surface area stimulated, the greater recruitment of a specific dermatomal distribution and the larger the contact area the more nerve fibers covered, increasing the opportunity for greater sensitivity. In a preferred embodiment of the invention therefore, a silver/silver chloride surface electrode with a contact area of about 2-4 inches is utilized, which is a larger surface area than conventional electrodes of about 0.9 cm ( 3/16- 12/16 inch) in diameter.

By means of the inventive software protocols the practitioner may adjust the stimulus applied to a stimulating site until optimization is achieved, enabling discernment of the exact loci for optimal stimulus. Thus, while dermatomal maps are known in the art, the inventive approach enables the prediction of exact loci within the dermatome for improved and reproducible data. Sites at which the stimulating modules are placed to elicit the dermatomal response may be specified by the practitioner by means of the inventive protocols.

After the stimulating modules are placed at the appropriate sites on the subject, the stimulating impulse is delivered to each selection site.

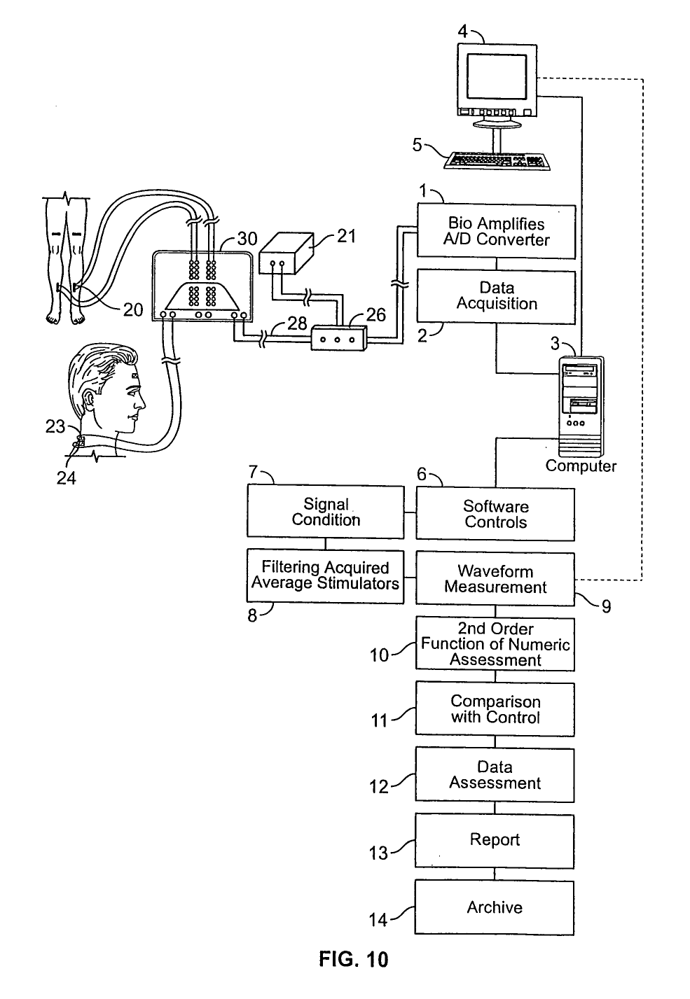

The inventors have identified a subcortical potential of lower amplitude over the posterior cervical spine, found to be a highly stable site for assessing absolute latencies and amplitudes of evoked potential waveforms when enhanced using the digital signal averaging techniques of the present invention (see (23, 24) in FIG. 10). Therefore, one aspect of the invention is that the subcortical recording site, as shown in FIG. 5 (60), produces superior and robust signals whether from right side or left side stimulation, eliminating issues concerned with the drifting neurological status of the brain as well as the effects of halogen-based anesthetic agents associated with the use of cortex-derived responses. Therefore in one aspect of the invention, a subcortical recording site is used exclusively.

FIG. 10 exemplifies and illustrates schematically an especially preferred embodiment of the invention by which stimulating modules are connected to the subject, recording processes are carried out, and measured responses are assessed and compared either statically or dynamically via the real-time processes as described herein. As described above and as it will be understood by those skilled in the art, the real-time neurophysiological assessment described herein may be conducted in a clinical or surgical setting. In the surgical setting, the practitioner or surgeon is herewith provided the ability to rapidly assess the collected data, which is then compared to a subject's prior baseline recordings, or to normals obtained from a non-symptomatic population. Surgical application of continuously comparing the measured responses in a subject undergoing an operative procedure allows real-time mixed and individual nerve root function to be evaluated dynamically, throughout the course of the procedure. In addition according to the method herein described, stimulation and recording is repeated serially at each site of interest, and subsequent latency readings compared to baseline or normal latency readings. Thus, recordings indicated via electrodes positioned at for example (23, 24) in FIG. 10 can be visually observed by the attending surgeon at a display screen such as (4) on FIG. 10.

In an exemplary mode, electrodes are placed on the body in an anatomical region where the subject is symptomatic. The nerve stimulation causes evoked potentials to be generated at the electrode sites. FIG. 11 shows an exemplary connection box (see also (30) in FIG. 10), designed to allow attachment of multiple stimulating and recording electrodes on a subject at one time, allowing up to 8-stimulating sites on each side of the subject, 8-left side and 8-right side for a total of 16, with two (cortical/sub-cortical) recording sites for a clinical evaluation. For a surgical evaluation stimulation electrodes can be placed 4-left side and 4-right side, as well as 4-EMG recording electrodes left side, and 4-EMG recording electrodes right side sites, with two (cortical/sub-cortical) recording sites, controlling the attachment of multiple stimulating and recording electrodes to the subject. Sites on the box (shown as rings in FIG. 11) correlate with electrode placement on the subject: each ring in the box is a female DIN receptor receiving the male end of the appropriate electrode. The box is designated to allow both recording and stimulating sites. Recording sites for electrodes placed over the posterior cervical spine become the subcortical recording site (FIG. 5 (60)). For upper extremities, only one electrode is generally used, but for lower extremities an additional recording site to the lumbar potential (FIG. 7 (61)) is optionally available as a frame of reference to add validity to latency measurements. In a preferred embodiment of the invention, there are a total of 16 available stimulating sites, up to 8 on each side of the subject, 8 on the left, and 8 on the right, with two cortical/subcortical recording sites. For surgical evaluations and operating room settings in general, for recording both nerve root potentials and electromyograph potentials, the hooded section of the box would provide for stimulating 4 left and 4 right side sites, as well as 4 recording left, and 4 recording right side EMG sites, with two (cortical/subcortical) recording sites.

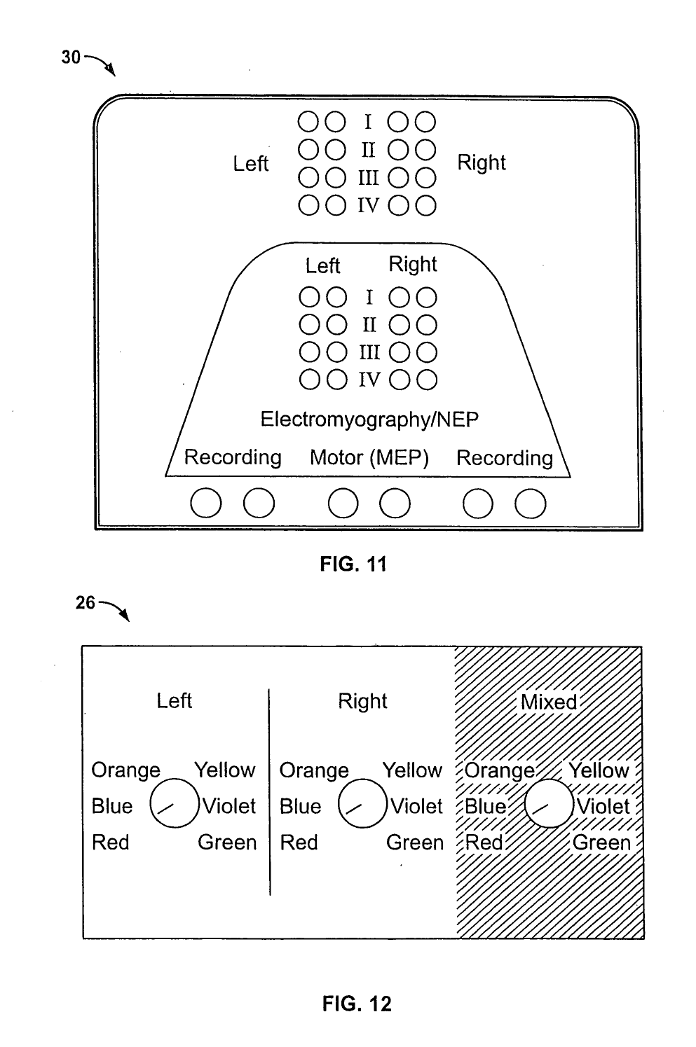

FIG. 12 shows an exemplary stimulus site selector switchbox (see also FIG. 10 (26)) designed to allow control of which sites are receiving a stimulus, where for example, red is site 1, (L1 left and right), blue is site 2, (L2 left and right), orange is site 3, (L3 left and right) and yellow is site 4, (L4 left and right), violet is site 5 (L5, left and right), and green is site 6 (S1 left and right). Mixed nerves or motor evoked potentials are stimulated in a left to right sequence, red-left, blue-right, orange-left, yellow-right, violet-left, and green-right. In a clinical setting, the sites may be for recording or for stimulus, whereas in an operating room setting, or intraoperatively, a total of 8 stimulus channels is used. In a mixed median response for example, red is L3, blue is L4, orange is R3, yellow is R4, and violet and green are reserved for motor stimuli.

Thus as described heretofore, a pair of electrodes (20) are placed on the leg at a stimulation site selector for the L4 dermatome and the generated potentials transmitted to the subject connection box (30). The transit time from a stimulus site to the recording electrodes placed over the posterior cervical spine (23, 24) is recorded at the subcortical recording site (see FIG. 5 (60)), from which a replicable conduction latency is obtained. In principle, a single unipolar electrode can be used to obtain a recording at the subcortical recording site, but as illustrated in FIG. 10, a pair of bipolar electrodes placed at the subcortical recording site approximately 2 cm apart is an optimal configuration in the inventive approach. The posterior cervical spine recording electrodes are connected to the subject connection box (30). Stimulation site selector (26) directs the electrical impulses for stimulating the left/right, dermatomal and mixed nerve responses sites to be stimulated with electrical impulses. Conductors (28) for carrying the impulse may be copper conductors, coaxial conductors, twisted shielded conductor pairs, or the other suitable conductors. The subject connection box (30) directs impulses from the stimulation site to the specific electrode attachments to the subject. The subject connection box (30) is schematically represented in FIG. 11. Electrical stimulation (current/mA) is applied by via electrical stimulator (21) and the stimulus site selector (26), shown in FIG. 12. Stimulating electrodes (20) placed on the subject, are connected to the subject connection box (30). Output signals from bio-amplifier/A-D converter (1) to the data acquisition unit (2) via a USB connector to a computer (3) are observed on display screen (4) to which the practitioner has access via keyboard (5). Computer (3) contains data buffers to which the recordings data is transported for later assessment and comparison, and first, second, third and forth data storage devices. Boxes (6)-(14) in FIG. 10 represent operations carried out by the computer in a preferred embodiment of the invention. For example, (6) represents software controls, (7) signal conditioning, (8) software controls for low to high frequency filtering of elicited recordings, (9) waveform measurement, (10) second order transport of assigned numeric values in which the process of comparing waveform data in one database with that of another is performed, (11) in which comparison to normals or control values is carried out, (12) in which assessment of compared data takes place, (13) in which a report is generated and (14) in which reports are archived.

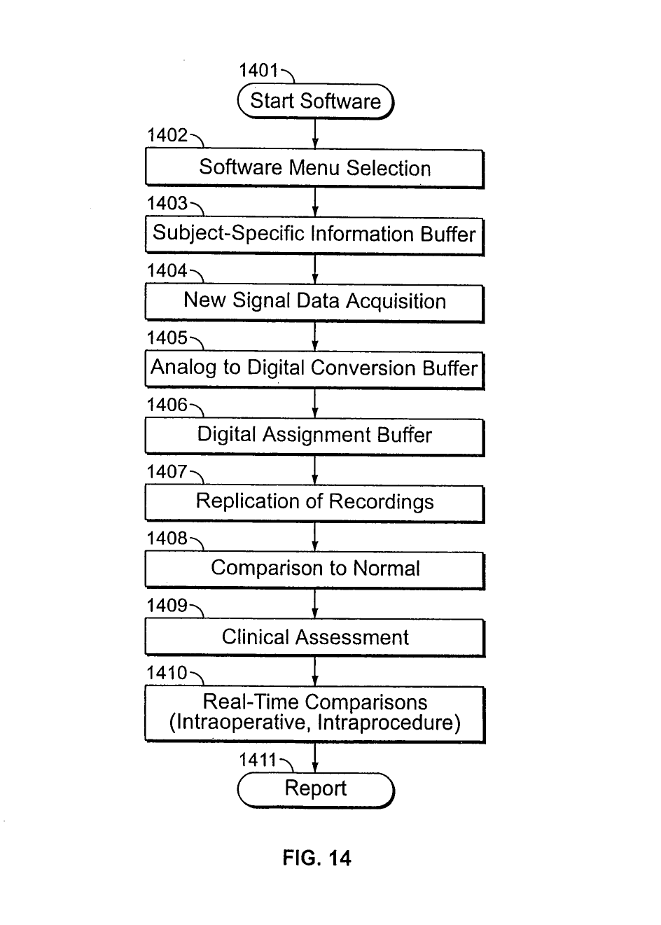

FIG. 14 depicts a flowchart of software operations for carrying out both clinical (static) settings, and intraoperative, or intraprocedural, settings in which a real-time neurological assessment of a subject being stimulated by an electrode at a dermatomal stimulation locus may be conducted as recordings are being made. It will be understood by those skilled in the art that the inventive approach assigns data being recorded to buffers for instant recall during the course of the procedure, and to permanent storage for archiving. It will also be understood by those skilled in the art that the inventive approach may have many variations on the substance of FIGS. 10 and 14 without departing from the spirit of the present inventive approach, which is to provide rapid and automatic real-time mathematical assessment of waveforms to provide dynamic and critical assessment and assurance to a practitioner during a procedure.

In FIG. 14, 1401 initiates the software means for carrying out the steps of the described approach, namely, eliciting an evoked response from a first stimulation site on a subject, receiving and amplifying a stimulation signal, and recording the waveform signal; automatically digitally converting the waveform signal and assigning numeric values for the absolute amplitude and absolute latency of the waveform; replicating the aforesaid to obtain a series of replicated digitally assigned waveform data for the given stimulation site; mathematically conditioning the series of replicated digitally assigned waveform data, obtaining a validated mean value for the series of replicated digitally assigned waveform data, and obtaining a comparison of the validated mean value with a protocol-specific datum and a subject-specific normal datum. At 1401, Icon Haris™ appears onscreen to navigate the user through the inventive system. 1402 is a user menu in which protocol options are presented for selecting a recording protocol, selecting and confirming stimulation site and proper electrode placement and the like. The practitioner inputs information relating to the particular procedure being initiated, such as for example, Uppers, Lowers, Clinical, Intraoperative, etc. At 1403, subject-specific information is loaded into a buffer for later access. Here, the practitioner is prompted to input subject-specific information such as subject history and stimulation and recording parameters. The software associates the subject-specific data with data for normals from a Normal Data Buffer. The normals buffer is populated as required by the practitioner by recording the appropriate neurological data obtained from a non-symptomatic population, or by inputting the data from known subjects, including but not limited to a test subject or subject prior to a procedure. Then follows stimulation and the recording of a waveform (in analog). The stimulation signal is received and amplified. At 1404, the new signal waveform data is allocated into a protocol selection/subject history-specific allocation in a first permanent storage, and here the analog waveform may be visualized via a display. At 1405, the now protocol-specific waveform data is loaded into a Conversion Buffer for analog to digital assignment. The data is then transported into a Digital Assignment Buffer (1406). At 1406, automatic assignment of absolute amplitude is made by measuring from Marker I, representing the first linear increase, to Marker II representing the peak of linear increase, giving an absolute digital value for the amplitude of the waveform in microvolts (μV). Automatic assignment of absolute latency is made by measuring the peak of linear aggression (Marker II), giving an absolute digital value for the latency in milliseconds (ms). At 1407, replications of recordings of the waveform are performed and the data stored in serial sequence using a first order mathematical function. Variations of the normal distribution of assigned absolute digital values of greater than 1 standard deviation (sd) are reported as skewed, with a correlation coefficient of 0.90 for validation of correlation. A mathematical conditioning algorithm is used to obtain a validated mean mathematical representation of the averaged response at the given stimulating and recording electrodes. The validated mean is then assessed for its absolute latency and for its absolute amplitude. Mean validated data is then available for comparisons to normal data, normal data being mathematically conditioned and obtained in a similar manner. As will be discussed below, the number of replications required for validated mean waveform data when the present inventive approach is conducted is far fewer than conventionally required. At 1408, using a second order mathematical function, comparisons to normal are made in which the replicated protocol/subject specific data in the Digital Assignment Buffer is compared to protocol/subject specific normal data in the Normal Data Buffer. At 1409, visual display of recorded values and normal values provides the practitioner with the ability to make a clinical assessment. A report of assessments of the comparisons between recorded waveform data to normal data generated with deviations noted is automatically generated.

At 1410, using a third order mathematical function, real-time comparisons are made in which the assigned validated digital values of recorded waveforms (protocol selection/subject history-specific) residing in the Digital Assignment Buffer are compared to normals in the Normal Data Buffer. Then serial comparisons are made as function of time in the Real-time Change Buffer throughout the course of the procedure. Variations are reported as skewed deviations ±1.0 sd. At 1411, a report is automatically generated comprising assessments of the comparisons of recorded values to normals and changes in recorded values noted as function of time. As before, visual display of the comparisons of recorded values and normal values as a function of time is provided to the practitioner throughout the course of the procedure.

A measured evoked response from a recording site is obtained and amplified to produce a robust waveform. After analog to digital conversion of the data, quick assessment of waveforms is made because the waveforms are placed in a digital format where they can be easily measured, saved and transported, for future use and comparison. A recorded response is cursor marked for visual inspection of the wave morphology, then saved and compared with a normal response. This process is continued in sequence until the end of the testing protocol. The mathematically summated tracings (signal averaging) of the physiological responses from the recording electrodes are time-locked to a given stimulus, and replicated for a determined number of responses. A mean mathematical representation is then presented as the averaged response at those recording electrodes to the given stimulus. The evoked response is then assessed for its absolute latency and absolute amplitude. Signal amplification reduces the signal to noise ratio, improving signal averaging. As a result, substantially fewer number of replications are needed to produce robust and reliable data than is conventionally required.

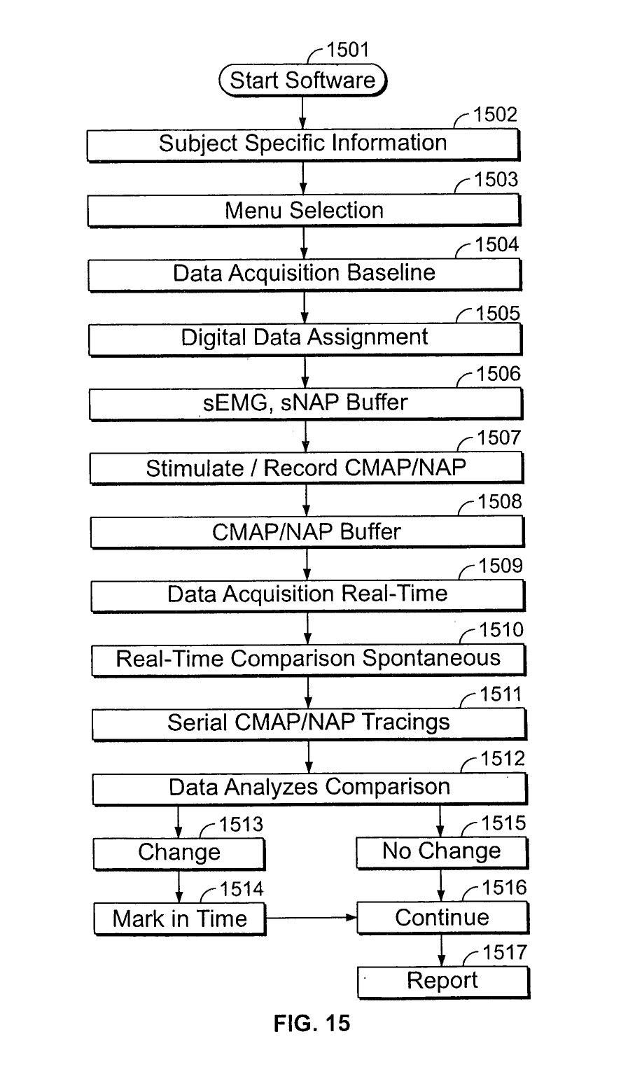

FIG. 15 depicts a flowchart of software operations for carrying out real-time monitoring of an at-risk nerve. (1501) indicates the start of the software; (1502) prompts for input of subject specific information (e.g. patient history, operative information, surgeon, date, time, hospital, etc) and, via menu selection (1503) procedural data input (e.g. select procedure/patient side, nerves/muscles to be monitored, recording montages, etc); (1504) indicates data acquisition baseline, start of recording of data, s EMG, sNAP; (1505) indicates digital data assignment, in which the data recorded in (1504) is placed into a comparison buffer including data analysis features; (1506) indicates spontaneous comparison buffers receiving the recorded sEMG, sNAP data displayed as baseline recordings, comparison windows are opened to obtain the next set of data; (1507) indicates obtaining of stimulus-originated data (stimulation can be mechanical or electrical), averaging the data, recording and assessing baseline CMAP and NAP tracings; (1508) indicates averaged comparison buffer—from which the recorded CMAP and NAP are displayed as baseline recordings and the comparison windows are opened to obtain the next set of data; (1509) indicates assessment of data acquisition real-time-baseline data and querying of the next real time comparison; (1510) indicates a spontaneous comparison window which records a determined segment of sEMG and sNAP data, sends the segment for comparison (2nd order comparison), and continues to record real time spontaneous data; (1511) indicates an averaged comparison window, in which at the users discretion another set of averaged CMAP and NAP are obtained, in which stimulation used can be mechanical or electrical; (1512) indicates serial data comparisons, in which each set of newly recorded data is compared to the baseline sets of data; (1513) denotes that a change from baseline latency, frequency or amplitude has occurred; (1514) indicates notation of the change that has occurred, its location, its type, and marks and reports the change; (1515) indicates no change; (1516) indicates continue recording; and (1517) indicates store and memorialize the entire tracing (burn cd).

Those skilled in the art will appreciate that the above may be carried out statically or dynamically, and may be carried out using a variety of nerves and muscles on a subject. Real-time assessments and comparisons are provided to the practitioner or surgeon for monitoring and guidance purposes, wherein waveforms obtained serially over the course of a procedure and dynamically assessed and compared in real-time. Serial comparisons of individual waveform data (frequency, latency and amplitude) may be made via any means of visual display of the buffered waveform data.

Comparisons may be made while recordings are taken with one or more normal or baseline recordings from the subject undergoing the procedure, or from an asymptomatic population, according to criteria selected by the practitioner. Baseline or normal latency control values may be obtained from a variety of different non-symptomatic population or mammalian subject sources with correction factors for height and limb length and limb temperature. The normal or control population database may be determined by each geographical location where the method is used, and may be selected according to criteria specified by the practitioner, such as for example species, race, gender, age, weight or height. Alternatively, a normal or control database may be obtained from a test subject or a subject, such as for example where measurements are made for experimental or clinical trial or research purposes, and the like. If measurements according to the inventive approach are being used to clinically or surgically evaluate an intervention, the control measurement may be made on a test subject or on a subject, prior to the intervention, where the test measurements are carried out on the same subject at or after the time of the intervention. If the measurements are being carried out to evaluate a medical instrumentation or develop such, or as part of a drug development platform, the measurements may be made on any number of control subjects and any number of different test subjects.

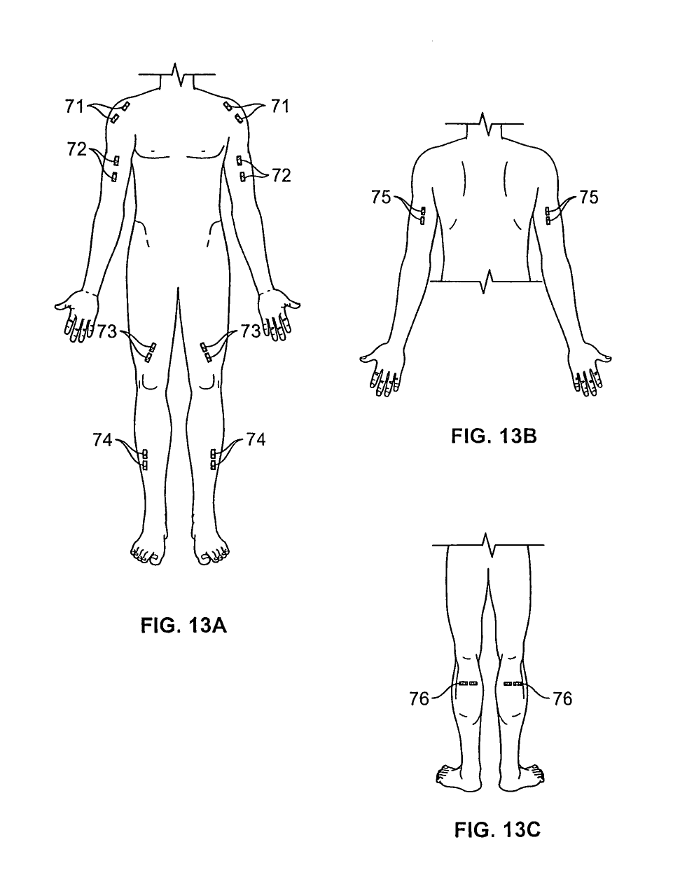

For example, assessment of wave presentations occurring in an electromyogram may be integrated with DSSEP data for determining the function of muscles innervated by the nerve root. Such activity may be used both as a marker for stimulus and as a marker for pathology. EMG activity is assessed in free-run format, using recording sites as shown in FIGS. 13A-C, comprising assessing a baseline waveform activity, then assessing a subsequent activity, wherein a transient increase in amplitude reflecting a muscle activity near a specific nerve root may be measured and correlated with a dermatomal evoked potential.

The approach may be used to assess the adequacy and safety of a nerve root decompression during spine surgery. During surgery, the inventive approach may be used to help prevent irreversible nervous system damage. Dynamic status of the nerve roots latency during decompression as described herein provides the surgeon with a real-time assessment of the adequacy of decompression. If the latency of waveforms is delayed, when compared to normal or control values, the surgeon or practitioner may suspect a pathological process at that specific root level. Pre-decompression latency delays intraoperatively, would therefore provide the surgeon with electrophysiological evidence of nerve root pathology and possible compression.

A dynamic alarm can provide early warning during a surgical procedure that would warn of possible physiological insult to a neural structure and help prevent a post-operative pathological presentation. Thus, the inventive approach can be used to improve the intraoperative efficacy of a surgical intervention and help evaluate the clinical presentation of the subject. For instance, when an evoked response is obtained after limb stimulation from, for example, a subcortical recording site, and a delayed response due to a pathological cause recorded, and upon removal of the pathology a new recording is obtained and compared in real-time with the previous recording and/or with a pre-existing normal response time, and found to be an improved response time, the surgeon is immediately prompted as to the efficacy of the action.

One aspect of the invention comprises a system comprising a computer, data acquisition devices and software-driven recording and comparison protocols, for comparing and assessing the recordings in real-time. Data from responses are transported into a series of buffers for immediate recall and processing, and may be stored in temporary and permanent data storage devices. Customized software enables the practitioner to automatically assign digital values for the absolute latencies and amplitudes of evoked potential waveforms while the recordings are taking place, automatically validate the waveform data, and dynamically compare and assess waveform data. Customized software provides and displays warnings of pathological changes as they occur which may be color-coded or may be provided in any other suitable alerting form, confirms improved changes, archives data, generates reports, as well as a diverse variety of further functions described herein.

In another aspect of the invention is provided an icon (Haris™) which is a virtual pointer or mouse appearing on the screen at the start to prompt or guide the user through every aspect of the customized software, such as for example taking subject history, helping select a protocol, confirming proper electrode placement, recording a sequence of responses, data analysis, determining baselines, displaying warnings of pathological changes, data archiving and generation of reports.

Following stimulation, waveforms are recorded and time-locked. To obtain a standard deviation, each impulse from a site is given a digital latency value in milliseconds (ms). The response is measured from the baseline to peak onset as the absolute latency. The peak is marked and saved as a comparative measured numeric representation, and the tracings summated or averaged.

In the inventive approach, mathematical signal enhancement is performed to produce robust waveforms in a fewer number of replications. Signal averaging is the mathematically summated tracings of the physiological responses from recording electrodes. The summated tracings are time-locked to a given stimulus (constant current mA/constant voltagen) (duration 0.2-1.0 ms). The tracing reflects in time (ms) the detection of the evoked response, a predetermined window in milliseconds in which the response is selected, between 50 ms upper extremities and 100 ms lower extremities. The tracings are replicated for a determined number of responses. A mean mathematical representation is then presented as the averaged response at those recording electrodes to the given stimulus. The evoked response is then assessed for its absolute latency, which is the first negative occurring wave morphology, following the first positive occurring wave morphology as a function of time.

Conventionally, speaking negative polarity is up. Waveforms are further assessed for amplitude, which is measured in μV (micro volts) from the beginning of the negative wave morphology to its greatest peak. The evoked response representation is then assessed for its absolute latency, which is the first negative occurring wave morphology following the first positive occurring wave morphology as a function of time for convention (where negative is up).

The tracings are further assessed for amplitude which is measured in microvolts (μV) from the beginning of the negative wave morphology to its greater peak. The nerve stimulation causes evoked potentials to be generated at various electrode sites. These generated potentials are transmitted to subject connection box (30). By measuring the transit time from the stimulus site to the desired recording electrodes over the posterior cervical spine, FIG. 10 (23,24), a replicable conduction latency is determined, and the measured evoked response converted to a waveform tracing as described. The A-D converter (FIG. 10 (1)) allows for a quick assessment on the reading of waveforms because the waveforms are placed in a digital format where they can be easily measured, saved and transported, for future use and comparison.

A program containing a second order transport function following a pre-determined recording protocol has been written to allow the performing of real-time comparisons within the program. In one embodiment, these may be performed by the practitioner via a user-interface. A recorded response is cursor marked for visual inspection of the wave morphology. It is then saved and compared with a normal response. This process is continued in sequence to the end of the testing protocol. The test comparisons of the recorded responses are compared to normal for the evaluation of latency and amplitudes.

The range of latencies (low to high in milliseconds) for upper and lower extremities, in male and female subjects respectively, is as follows:

for upper extremities: male 31.0-39.0, female 30.0-38.0

for lower extremities: male 51.2-58.0, female 50.6-57.0

The following formula mathematically represents each bilateral upper and lower dermatomal site, where Rx=the response recorded, Ry=the known normal value for the given response, and Rx±Ry is <no reported change> or is, for upper extremities:
male 3.0 ms=1.0 sd (where 10 μV=1.0 sd)
female 2.7 ms=1.0 sd (where 8 μV=1.0 sd)

for lower extremities:
male 3.2 ms=1.0 sd (where 12 μV=1.0 sd)
female 2.8 ms=1.0 sd (where 0.9 μV=1.0 sd)

Discrepancies in latencies and amplitude have standard deviation (sd) increments from zero sd to a maximum of 3.0 sd, where normals are taken from a population database determined by each geographical location in which the method is used.

After averaging and layering, the waveforms are defined. Waveform tracings are replicated for a determined number of replications. Conventionally, some 2000 samples must normally be conducted. With the modifications according to the present invention in the recording techniques used (larger stimulation surface areas of 2-4 inches, use of the subcortical recording area, signal amplification, fewer replications with digital averaging) dermatomal nerve root response of a high technical quality is recorded with acceptable replication with between 100 and 200 recording trials conducted. According to the inventive method, a conduction latency in milliseconds (ms), is recorded from a stable site that functionally amplifies a recording site of the absolute latency ±10 ms from the initial marked absolute latency. The signal averaging techniques are thereby enhanced to expedite the recording process, from the measured first absolute latency, a range of −10 ms to +10 ms (where the window for upper extremities is between zero and 50 ms using regular equipment) is established as a range of subsequent acceptable recorded responses to be the summated mean latency. With this step, replicate recordings of dermatomal responses can be made rapidly and of high technical quality. In the inventive approach, the standards of normality are more rigorously obtained.

For example, for the C5 cervical site, where C5x=the recorded value for that site, C5y=a corrected normal value for that site with a known ± variance, C5y±C5x=C5z, and C5z=difference±absolute latency (with correction of the known recorded value of absolute latency±the known variance of that latency). If C5z is a numerical representation of a + as a latency delay, if the delay first exceeded the known positive variance in the recorded normal when compared, then it is reported as a representation of standard deviations from normal. The normal value recorded for a C5 subcortical latency is 14.7 ms±2.5 ms on the left side and 15.4 ms±1.0 ms on the right. If a C5z represents a latency delay, it is greater than 14.9+0.9=16.6 and 14.4+1.0=15.4 ms, respectively. With approximately 128 collected signal enhancement comparisons for a range of standard deviations, for a given population, C5 as 3.1 ms represents a single standard deviation. If the correct dermatomal stimulating electrodes are connected to the correct site, the site will be stimulated and compared to the normal, and the findings reported as within either a normal range or abnormal range in terms of the number of standard deviations from the normal.

In a preferred embodiment, the inventive device is configured to include a latency fail-safe feature as referred to above that alerts the practitioner if within a given set of recording data. For example, in C5, C6, C7, C8, left and right side, there might appear to be non-linear representations. For example, C5 responses should occur at approximately 14-16 ms, C6 at approximately 20-23 ms, C7 at approximately 21-23 ms, and C8 at approximately 22-24 ms. Thus, if the site at C5 has bilateral representations of between 22 and 24 ms, the software system first prompts for confirmation of electrode placement before assuming bilateral delays.

In an especially preferred embodiment of the inventive approach, the signal-to-noise ratio (s/n) is increased by means of a biosignal amplifier for EEGs (e.g. Dual Bio-Amp™, AD Instruments Pty Ltd), for electromyogram, EMG (e.g. g.tec™ Guger Technologies OEG) in conjunction with data acquisition hardware such as Power Lab™ AD Instruments Pty Ltd as the recording device. The bio-amplifier (Dual Bio Amp™) used in the inventive approach reduces the signal to noise ratio, improving signal averaging.

In theory, noise of an individual response is random with respect to the stimulus, thus the net sum of noise following the stimulus increases as n increases, where n=number of trials of time-locked recordings. The evoked response follows the same time course after each stimulus, thus there is no cancellation of this signal as responses are summated. Instead, the amplitude of the evoked response increases in direct proportion to the number of stimuli (n), and by increasing n, one is able to enhance the signal to noise ratio by the factor: n/√n. By improving the signal to noise ratio, the total number of recording trials needed is reduced without skewing amplitude determinations.

In another especially preferred embodiment, FIGS. 13A-C illustrate recording sites via which recordings of spontaneous free-run EMG data by means of which verification of nerve roots may be made, providing another level of physical correlation to identify the muscle that nerves innervate. In one embodiment, the EMG activity is first assessed as a baseline waveform, then subsequently as a subsequent waveform activity. The transient increase in amplitude reflecting a muscle activity near a specific nerve root is measured and may be correlated with a dermatomal evoked potential. Muscle physiology reflecting the nerve root function is evaluated by inserting a needle into an appropriate muscle and observing both visual and auditory electrical muscle potentials. The amplifier is turned on and spontaneous activity may then be viewed and heard, or may be received in any other suitable electronic form. Recording electrodes are placed in the muscle via needle electrodes or over the muscle via surface electrodes, in the place where the muscle is to be evaluated. As a stored ratio of amplitude latency for a known duration, these stored samples are converted into a mathematical representation. The baseline free run EMG activity is then entered for a known muscle i.e. deltoids, biceps, triceps, bilaterally. Transient increases in amplitude for short durations in specific muscle on a specific side identifies physical activity near a specific nerve root which can be correlated with dermatomal evoked potential studies during surgery. FIG. 13A shows the positions of the deltoid (71), the bicep (72), and the quadriceps (73) recording sites. FIG. 13B shows the positions of the triceps recording site (75), and FIG. 13C, the position of the gastrocnemius recording site (76). Near nerve activity in the muscle that would show a transient linear increase, compared to a baseline recording, is then correlated with the nerve root responses, for example: deltoid C4, C5 roots in that side, bicep C5, C6, and triceps C6, C7. The root evaluation for the noted muscle would determine if there is a change of the root latency. If a change in the DSSEP waveform latency is noted, this could be correlated with changes in muscle potentials caused by root irritation, thereby providing a real-time “cross-check correlation”. One root would be identified providing irritation in the EMG, which could then further identify a specific level.

In a preferred embodiment, the inventive approach shown in FIG. 10 is followed but including use of the recording sites shown in FIGS. 13A-C. A motor complex waveform may be identified from the background EMG activity and then used as a marker for the stimulus. The software system records several hundred milliseconds of signal following its occurrence. It then averages the intervals of occurrence to determine a baseline recording. Pathological occurrences affecting the EMG manifest as transient shorter interval train of random amplitude inter-peaks, latency are recognized as a near nerve root signal. This recognition identifies a change to the baseline intervals and a report may be generated showing the physical activity being close to a nerve, or nerve root. The trained responses of varying amplitude and random inter-peaks when compared to baseline intervals identify a mechanical stimulation, that is, pressure or traction on a nerve root. A burst response of considerable amplitude increase and a wide inter-peak latency identifies a direct contact with an innervated structure. The frequent and/or persistent occurrence of burst activity may be correlated with neurological defects in the innervated musculature due to nerve root pathology noted. A preferred embodiment of the invention is configured to respond to these actions by provoking a warning alert, which is prompted as an audible or color change to the recorded EMG tracing. A first reading which may act as a control reading or a baseline reading is made at the desired EMG site and the waveforms are saved for comparison when subsequent testing is performed, and the subsequent testing performed during surgery in real-time.

### EXAMPLE 1

The invention provides a multichannel neurophysiologic monitoring system and protocol to intraoperatively assess an at risk nerve function, such as for example, the sciatic nerve during a RTHA surgical procedure. To address the incidence of occurrence of post-operative nerve palsy or damage, the inventive multichannel channel neurophysiologic monitoring system may be used to assess at-risk nerve function during a surgical procedure, spontaneously elicited electromyography (sEMG) and mechanically elicited (mEMG), electrically elicited compound motor action potential (eCMAP), and electrically elicited nerve action potential (eNAP) and spontaneous nerve action potential (sNAP) is interpreted in real-time during surgical exposure of the at risk nerve. The surgical approach is redirected until all firings subside spontaneously.

In an exemplary mode, where the sciatic nerve is at risk, sEMG, mEMG and eCMAP is recorded from musculature such as for example, the quadriceps, tibialis anterior, gastrocnemius, and EHL musculature, eNAP and sNAP is obtained for example, from the medial and lateral femoral cutaneous, peroneal and posterior tibial nerves. A bipolar wan has been designed for use by the surgeon to locate the at risk nerve during exposure, then repeat elicited responses during and following procedure such as for example, placement of acetabular and femoral components during RTHA, as well as prior to closure.

During RTHA, sEMG and mEMG recordings may be obtained for example at the quadriceps, the tibialis anterior, the gastrocnemius, and the EHL muscle, sNAP, mNAP and eNAP recordings may be obtained over a nerve selected from the group consisting of the medial femoral cutaneous, the lateral femoral cutaneous, the peroneal, the posterior tibial, and eCMAP recordings are obtained at a muscle selected from the group consisting of the quadriceps, the tibialis anterior, the gastrocnemius, and the EHL muscle. Those skilled in the art will recognize that the inventive approach is not necessarily limited to the above mentioned nerves and muscles, and listed at-risk nerves cited herein are exemplary not exhaustive.

Examples of at risk nerves during wrist replacement surgery are the median nerve, which may be monitored for example by eCMAP recordings at the abductor pollicis brevis (thenar eminence), the ulnar nerve which may be monitored by eCMAP recordings at the first dorsal interosseous and the abductor digitis minimi, and the radial nerve monitored by eCMAP recordings at the extensor indicis proprius and abductor pollicis longus. In conjunction, eNAP and mNAP monitoring of, for example, the median nerve, ulnar nerve and radial nerve is conducted. sEMG and mEMG may be carried out on, for example, the first dorsal interosseous, the abductor digiti minimi, the extensor indicis proprius, abductor pollicis longus and abductor pollicis brevis (thenar eminence).

Examples of at risk nerves during elbow replacement surgery are the median nerve, which may be monitored for example by eCMAP recordings at the abductor pollicis brevis (thenar eminence), the ulnar nerve which may be monitored by eCMAP recordings at the abductor digiti minimi, first dorsal interosseous and the adductor pollicis, and the radial nerve monitored by eCMAP recordings at the extensor indicis proprius and abductor pollicis longus. In conjunction, eNAP and mNAP monitoring of the median nerve, ulnar nerve and radial nerve may be conducted. sEMG and mEMG monitoring may be carried out on, for example, the abductor pollicis brevis, first dorsal interosseous, abductor digiti minimi, extensor indicis proprius, abductor pollicis longus, abductor pollicis brevis, pronator teres, flexor pollicis longus, flexor carpi radialis, flexor carpi ulnaris and extensor digitorum communis.

During shoulder replacement surgery, stimulation can be made at the operative site or the Erbs point. The at-risk nerves are, among others, the median, ulnar, radial, axillary, musculocutaneous and suprascapular nerves. eCMAP recordings can be obtained from the abductor pollicis brevis (median nerve), the abductor digitis minimi and first dorsal interosseous (ulnar nerve), extensor indicis proprius and abductor pollicis longus (radial nerve), deltoid (axillary nerve), bicep (musculocutaneous nerve) and infraspinatus muscle (suprascapular nerve). In conjunction, eNAP and mNAP monitoring of the median nerve, ulnar nerve, radial nerve, axillary nerve, musculocutaneous and suprascapular nerves may be conducted. sEMG and mEMG monitoring may be carried out on, for example, the abductor pollicis brevis, first dorsal interosseous, abductor digiti minimi, the tricep, bicep, deltoid, infraspinatus, extensor indicis proprius, abductor pollicis longus, abductor pollicis, pronator teres, flexor pollicis longus, flexor carpi radialis, flexor carpi ulnaris and extensor digitorum communis.

During ankle replacement. surgery, the at-risk nerves are the deep peroneal, superficial peroneal and posterior tibial nerves, monitored by eCMAP recordings of the extensor digitorum brevis, the peroneus longus, the abductor digiti minimi (foot) and the adductor hallucis. In conjunction, eNAP and mNAP monitoring of the deep peroneal, superficial peroneal, posterior tibial and sural nerves, and sEMG and mEMG of extensor digitorum brevis, peroneus longus, abductor digiti minimi and adductor hallucis is carried out.

During knee replacement surgery, stimulation may be carried out at the operative site and/or at the sciatic nerve. An exemplary procedure comprises monitoring the at risk posterior tibial and common peroneal nerves via eCMAP recording of the abductor digiti minimi and extensor digitorum brevis, monitoring the deep peroneal, superficial peroneal, posterior tibial, and sural nerves by eNAP and mNAP recordings, and by sEMG and mEMG monitoring of the extensor digitorum brevis, peroneus longus, abductor digiti minimi, adductor hallucis, tibialis anterior, medial gastrocnemius and lateral gastrocnemius.

### EXAMPLE 2

The described monitoring methods and systems are readily adapted for assessing sciatic nerve function segmentally during a revision total hip arthroplasty (RTHA) surgery in which the sciatic nerve is an at-risk nerve. In an operative site where visualization of an at-risk nerve such as the sciatic nerve is difficult or impossible, elicited responses are used to locate the nerve, and monitor its integrity and function serially throughout the procedure. During and throughout the procedure, the sciatic nerve is serially located and serially electrically stimulated with a bipolar wand for monitoring of the functionality of the nerve.

A baseline spontaneously occurring electromyography (sEMG), and a baseline spontaneously occurring nerve action potential (sNAP) are recorded in the subject undergoing a RTHA surgical procedure. The firing rate, or frequency, of the spontaneous activity is converted to digital data for subsequent comparisons. The baseline data can be obtained from the patient at any time prior to any event that is potentially invasive or perturbing of baseline activity in that subject, typically before the start of surgery.

During and throughout the procedure, for example a hip procedure, mechanically elicited (surgical instrument movement or insult) electromyography (mEMG) and mechanically elicited (mNAP) recordings are serially obtained, the obtained waveform latency and amplitude data being converted in real-time to digital data for analysis and comparison.

Upon stimulation of the sciatic nerve with the bipolar wand, electrically elicited compound muscle action potential (eCMAP) and electrically elicited nerve action potential (eNAP) are recorded at the various musculature and nerve loci, the obtained waveform latency and amplitude data being converted in real-time to digital data. At selected intervals during the procedure, at the discretion of the surgeon, sciatic nerve stimulation is serially repeated, with the obtained waveform latency and amplitude data being converted in real-time to digital data for real-time analysis and comparison.

sEMG and mEMG recordings are, for example, obtained at the quadriceps, the tibialis anterior, the gastrocnemius, and the EHL muscle. sNAP, mNAP and eNAP recordings are obtained over the peroneal and posterior tibial nerves. SNAP and mNAP recordings are also, for example, obtained fro the medial and lateral femoral cutaneous nerves. eCMAP recordings are, for example, obtained at quadriceps, the tibialis anterior, the gastrocnemius, and the EHL muscle.

Baseline and real-time trend data is displayed in windows that individually show the firing rate or frequency (in the case of spontaneously occurring sEMG and sNAP), amplitude and latency of the obtained waveforms. When compared to baseline the firing rate of the sEMG or sNAP changes (increase or decrease) it is presented as a electrophysiological change. Frequency, amplitude and latency are each displayed with respect to time, and their analog data converted in real-time to digital data that provide a series of real-time outputs displayed and compared with respect to time. Evaluations of spontaneously occurring EMG and NAP with respect to baseline recordings are presented in real-time. Latency and amplitude changes are evaluated in real-time. Deviations from baselines or set norms are audibilized and provide alerts to the surgeon and practitioners.

The flow of data capture by the system may be understood by recourse to FIG. 15. At the beginning of the procedure subject specific information such as patient history, operative information, surgeon, date, time, hospital are input into the system. Via a user interface, menu selection makes available procedure options, nerves and muscles to be monitored, and recording montages. Data acquisition begins with recording of baseline data for sEMG and sNAP. A digital data assignment step places the data recorded into a comparison buffer having data analysis features. In a spontaneous comparison buffer, the recorded sEMG and sNAP are displayed as baseline recordings and the comparison windows are opened to obtain the next set of data. During stimulation, which may be either mechanical or electrical, serially obtained recordings are averaged, and stored. Baseline eCMAP and eNAP tracings are assessed. In the averaged comparison buffer—the recorded eCMAP and eNAP are displayed as baseline recordings and the comparison windows are opened to obtain the next set of data. Data acquisition real-time-baseline data is assessed and the next real time comparison is queried. Spontaneous comparison windows record a determined segments of sEMG and sNAP data, send the segments for comparison (2nd order comparison), and continue to record real-time spontaneous data. An averaged comparison window, at the user's discretion, displays another set of averaged obtained CMAP and NAP, stimulation being mechanical or electrical. In a serial data comparisons buffer, each set of newly recorded data is compared to the baseline sets of data. If a change from baseline latency, frequency or amplitude has occurred, the change is indicated and displayed, with a noting that the change has occurred, its location and type being marked, and the change reported. Recording continues throughout the procedure. The entire tracings throughout the procedure are stored and memorialized. In a group of 2,112 revision total hip surgeries, the results were as described in the following.

Prior to monitoring the incidence of post operative neurological deficits was 12%. Post operative foot drop, medical literature identifies 3-20% incidence of occurrence. During the 2112 cases, 1,435 electrophysiological warnings were identified, 912 electromyographic changes (602 firing increases, 256 latency changes, 54 amplitude changes) when compared to baseline, 523 neurogenic changes (411 near nerve changes, 59 latency changes, 53 amplitude changes). During the neurophysiological changes, the surgeon assessed what dynamic situation was occurring i.e., exposure, retraction, drilling, cup/stem placement, stretch, and waited for neurophysiological resolution of the electrophysiological presentation. The preponderance of neurophysiological warnings occurred during retraction, then cup preparation/placement, then stretch (limb lengthening), several significant events occurred during exposure and closing that were of complete surprise to the surgeon. Of the 2,112 monitored surgeries, no postoperative neurological deficits were noted.

The approach may be used for other surgeries performed on hip, shoulder, knee, wrist, ankle and the like, using the appropriately selected musculature and nerves to record at.

In the instant methods and systems, spontaneously occurring EMG and NEP activity, and elicited EMG, NEP and CMAP responses are processed by means of software via a computer processor and at least temporary memory buffers. Spontaneous activity and elicited responses are directed to real-time trend displays in communication with the processor and the buffers, at which displays practitioners may view analog and digital data with respect to time. The data consists of amplitude and latency waveforms in both analog and digital forms. The system is also configured to provide alerts running throughout the procedure, in which deviations from norms are audibilized as well as noted onscreen. The subject is assessed at optionally varying times such as, when at rest before a procedure, during a procedure but before an invasive step, during a procedure following a first invasive step, prior to instrumentation, and post instrumentation. Recording electrodes may be placed on the subject utilizing a stocking for specific montage arrangement.

It will be understood by those skilled in the art that other embodiments are possible, and that the inventive approach may be applied during a surgical or non-surgical manipulative procedure.

For example, another application of the inventive methods and systems is is a non-invasive diagnostic device or approach, as a fairly rapid and non-invasive means or diagnostic procedure for surgical management and/or for determining if surgery would be indicated to verify surgical procedures or ascertain conditions of the body comprising for example pathologies of various locations of the body such as back, cervical spine, anterior spine, head, shoulders, pelvis, hip, leg, knee, etc. and surgeries such as for example spine surgery, hip surgery, vascular surgery (carotid, aorta etc.), tumor removal, etc. The inventive approach can be used in the doctor's office or in any clinical setting to aid evaluation of complaints such as for example back, hip, and leg problems involving compression of nerves and nerve roots, including but not limited to chronic or acute, pain, numbness, tingling, pressure, weakness, discomfort, located for example in the neck, back, hip, buttock, groin, shoulder, arm, hand, finger, leg, shin, calf, foot, toe, due to illness, trauma or accident. The approach may also be used to correlate with clinical data from x-ray, MRI, CT scan, electromyogram, steroid injection, or a drug or other therapy or intervention.

Further, it will be obvious that the inventive approach may provide a non-invasive means during a procedure upon a mammalian subject for development and/or testing of a medicament, or pharmaceutical and the like, or for the testing of an instrumentation or device during the design and development of medical instrument technology.

It should be understood that the present invention as described in the foregoing, may incorporate various changes, substitutions and alterations without departing from the spirit and scope of the invention as defined by the appended claims.

1. A method for monitoring and assessing the function of an at-risk nerve in a subject during a surgical procedure, comprising the steps of:
a. obtaining a baseline spontaneously occurring electromyography activity (sEMG) recording; then serially, throughout the procedure, obtaining a plurality of mechanically elicited electromyography activity (mEMG) recordings; and therefrom obtaining a plurality of real-time mEMG/sEMG comparisons between the serially obtained mEMG recordings and the sEMG recording;
b. obtaining a baseline spontaneously occurring nerve action potential (sNAP) recording; then serially, throughout the procedure, recording a plurality of mechanically elicited nerve action potential (mNAP) recordings; and therefrom obtaining a plurality of real-time mNAP/ sNAP comparisons between the serially obtained mNAP recordings and the sNAP recording;
c. serially, throughout the procedure, electrically stimulating a (sciatic) nerve portion and following each act of stimulating, obtaining a plurality of electrically elicited compound muscle action potential (eCMAP) recordings, and a plurality of electrically elicited compound nerve action potential (eNAP) recordings; and therefrom obtaining a plurality of real-time eCMAP/eCMAP comparisons between the serially obtained eCMAP recordings and a plurality of real-time eNAP/eNAP comparisons between the serially obtained eNAP recordings,
wherein the obtained mEMG, sEMG, mNAP, sNAP, eCMAP, eNAP recordings, and the mEMG/sEMG, mNAP/sNAP, eCMAP/eCMAP and eNAP/eNAP comparisons are displayed by means of one or more of a plurality of real-time trend displays, and a processor adapted and configured to receive and store in a plurality of real-time data acquisition buffers the obtained mEMG, sEMG, mNAP, sNAP, eCMAP, eNAP recordings, and receive and store in a plurality of real-time comparison buffers the mEMG/sEMG, mNAP/sNAP, eCMAP/eCMAP and eNAP/eNAP comparisons, said processor being in communication with said real-time trend displays, and
wherein said real-time trend displays comprise: one or a plurality of baseline data windows wherein one or more baseline data are displayed; one or more amplitude waveform windows wherein one or more amplitude waveforms are displayed with respect to time; one or a plurality of latency waveform windows wherein one or more latency waveforms are displayed with respect to time; one or a plurality of frequency waveform windows wherein one or more frequency waveforms are displayed with respect to time; and one or a plurality of real-time digital value analysis windows, wherein recorded analog data converted to digital values are displayed with respect to time, said digital value analysis windows providing a plurality of evaluations in real-time.

2. The method of claim 1, wherein the procedure is performed upon a hip, a shoulder, a knee, a wrist, an elbow or an ankle.

3. The method of claim 1, wherein the at-risk nerve is a nerve selected from the group consisting of the sciatic, the median, the ulnar, the radial, the axillary, the musculocutaneous, the suprascapular, the deep peroneal, the superficial peroneal, the posterior tibial, the sural, and the common peroneal.

4. The method of claim 1, wherein the sEMG and mEMG recordings are obtained at one or more of a muscle selected from the group consisting of the quadriceps, the tibialis anterior, the gastrocnemius, the medial gastrocnemius, the lateral gastrocnemius, the extensor hallucis longus, the abductor, the abductor pollicis brevis, the thenar eminence, the first dorsal interosseous, the extensor indicis proprius, the abductor pollicis longus, the abductor digiti minimi, the pronator teres, the flexor pollicis longus, the flexor carpi radialis, the flexor carpi ulnaris, the extensor digitorum communis, the tricep, the bicep, the deltoid, and the infraspinatus.

5. The method of claim 1, wherein the sNAP, mNAP and eNAP recordings are obtained over one or more of a nerve selected from the group consisting of the medial femoral cutaneous, the lateral femoral cutaneous, the peroneal, the posterior tibial, the median, the ulnar, the radial, the brachial plexus, the axillary, the musculocutaneous, the suprascapular, the sural, the deep peroneal, and the superficial peroneal.

6. The method of claim 1, wherein the eCMAP recordings are obtained at one or more of a muscle selected from the group consisting of the quadriceps, the tibialis anterior, the gastrocnemius, the extensor hallucis longus, the abductor pollicis brevis, the thenar eminence, the first dorsal interosseous, the abductor pollicis, the extensor indicis proprius, the abductor pollicis longus, the abductor digiti minimi, the first dorsal interosseous, the axillary deltoid, the musculocutaneous, the suprascapular, the extensor digitorum communis, the tricep, the extensor digitorum brevis, the peroneus longus, the abductor digiti minimi and the abductor hallucis.

7. The method of claim 1, wherein one or more of the comparisons occurring with respect to time are audibilized.

8. The method of claim 1, wherein said baseline sEMG recording and said baseline sNAP recording are obtained at a time selected from the group consisting of: before a procedure, during a procedure but before an invasive step, during a procedure following a first invasive step, prior to instrumentation, and post instrumentation.

9. The method of claim 1, wherein one or more recordings are obtained via wireless electrodes.

10. The method of claim 1, wherein the at-risk nerve is located and stimulated with a bipolar wand.

11. The method of claim 10, wherein the bipolar wand is wirelessly powered.

12. The method of claim 1, further comprising using a montage stocking for placement of electrodes, wherein the montage stocking has positioned apertures corresponding to a specific electrode montage, wherein the electrodes are placed on the surface or just beneath the surface of the skin of a subject at the positions of the apertures.

13. The method of claim 1, further comprising using a multi-channel neurophysiologic monitoring system configured to control a plurality electrodes.

14. The method of claim 13, wherein the neurophysiologic monitoring system comprises wireless recording electrodes.

15. A system for monitoring and assessing the function of an at-risk nerve in a subject during a surgical procedure, comprising means for carrying out the method of claim 1.

16. The system of claim 15, wherein the procedure is performed upon a hip, a shoulder, a knee, a wrist, an elbow or an ankle.

17. The system of claim 16, wherein the at-risk nerve is a nerve selected from the group consisting of the sciatic, the median, the ulnar, the radial, the axillary, the musculocutaneous, the suprascapular, the deep peroneal, the superficial peroneal, the posterior tibial, the sural, and the common peroneal.

18. The system of claim 16, wherein the sEMG and mEMG recordings are obtained at one or more of a muscle selected from the group consisting of the quadriceps, the tibialis anterior, the gastrocnemius, the medial gastrocnemius, the lateral gastrocnemius, the extensor hallucis longus, the abductor, the abductor pollicis brevis, the thenar eminence, the first dorsal interosseous, the extensor indicis proprius, the abductor pollicis longus, the abductor digiti minimi, the pronator teres, the flexor pollicis longus, the flexor carpi radialis, the flexor carpi ulnaris, the extensor digitorum communis, the tricep, the bicep, the deltoid, and the infraspinatus.

19. The system of claim 16, wherein the sNAP, mNAP and eNAP recordings are obtained over one or more of a nerve selected from the group consisting of the medial femoral cutaneous, the lateral femoral cutaneous, the peroneal, the posterior tibial, the median, the ulnar, the radial, the brachial plexus, the axillary, the musculocutaneous, the suprascapular, the sural, the deep peroneal, and the superficial peroneal.

20. The system of claim 16, wherein the eCMAP recordings are obtained at one or more of a muscle selected from the group consisting of the quadriceps, the tibialis anterior, the gastrocnemius, the extensor hallucis longus, the abductor pollicis brevis, the thenar eminence, the first dorsal interosseous, the abductor pollicis, the extensor indicis proprius, the abductor pollicis longus, the abductor digiti minimi, the first dorsal interosseous, the axillary deltoid, the musculocutaneous, the suprascapular, the extensor digitorum communis, the tricep, the extensor digitorum brevis, the peroneus longus, the abductor digiti minimi and the abductor hallucis.

21. The system of claim 16, wherein one or more of the comparisons occurring with respect to time are audibilized.

22. The system of claim 16, wherein said baseline sEMG recording and said baseline sNAP recording are obtained at a time selected from the group consisting of: before a procedure, during a procedure but before an invasive step, during a procedure following a first invasive step, prior to instrumentation, and post instrumentation.

23. The system of claim 16, wherein one or more recordings are obtained via wireless electrodes.

24. The system of claim 16, wherein the at-risk nerve is located and stimulated with a bipolar wand.

25. The system of claim 16, wherein the bipolar wand is wirelessly powered.

26. The system of claim 16, further comprising using a montage stocking for placement of electrodes, wherein the montage stocking has positioned apertures corresponding to a specific electrode montage, wherein the electrodes are placed on the surface or just beneath the surface of the skin of a subject at the positions of the apertures.

27. The system of claim 16, further comprising using a multi-channel neurophysiologic monitoring system configured to control a plurality electrodes.

28. The system of claim 27, wherein the neurophysiologic monitoring system comprises wireless recording electrodes.

29. The system of claim 27, further comprising a subject connection means connected between the subject and the computer and comprising a plurality of receptor sites for inserting multiple stimulating and recording modules, wherein the receptor sites for inserting stimulating modules correlate with placement of stimulating modules on the subject; and wherein the receptor sites for inserting recording modules correlate with placement of recording modules on the subject.

30. The system of claim 27, further comprising a stimulus switchbox means connected between the subject connection means and an AND converter means, wherein the stimulus switchbox means provides for instrumental control of a plurality of recording and stimulating modules.

31. The system of claim 27, further comprising a software means for generating a deviation from normal warning signal via a visual, audible or electronic means.

32. The system of claim 27, further comprising a software means for providing and displaying an icon on a computer screen responsive to a command by a computer user, wherein the icon appears on the screen and prompts a user to select an option consisting of take a subject history, select a recording protocol, confirm proper module placement, input parameters, record a sequence, analyze data, archive data, or generate a report.
